(19) United States
(12) Patent Application Publication
(10) Pub. No.: US 2026/0315156 A1
(43) Pub. Date: Oct. 15, 2026

(54) APPARATUS AND METHOD FOR REMOVING CIRCULAR TRACKERS ATTACHED TO A TRACKING ARRAY

(71) Applicant: GLOBUS MEDICAL, INC., AUDUBON, PA (US)

(72) Inventor: Timothy J. Blackwell, Ft. Pierce, FL (US)

(21) Appl. No.: 19/465,329

(22) Filed: Jan. 30, 2026

Related U.S. Application Data
(63) Continuation of application No. 17/670,062, filed on Feb. 11, 2022, now Pat. No. 12,544,146.

(51) Int. Cl. A61B 34/20; A61B 90/00

(52) U.S. Cl. CPC A61B 34/20; A61B 90/00

(57) ABSTRACT

A tool for removing circular trackers attached to tracking array posts. The tool includes a tubular body and a driver tube sized to be received in the tubular body. The distal portion of the tubular body and driver tube has respective first and second apertures that are circumferentially partially open in a lateral direction for receiving the circular tracker. The two apertures cooperate with each other such that when the second aperture is rotated relative to the first aperture, the circular tracker is enclosed within the driver tube and the tubular body for removal without pulling on the tracking array or the post.

### CROSS-REFERENCE TO RELATED APPLICATIONS

This application is a divisional application of U.S. Patent Application No. 17/670,062, filed Feb. 11, 2022, the contents of which is incorporated by reference herein in its entirety for all purposes.

### FIELD

The present disclosure generally relates to optical tracking systems and more particularly a tool for removing circular trackers attached to a tracking array.

### BACKGROUND

Tracking arrays attached to a patient are used to track the patient body parts in navigation assisted surgeries. Typically, the circular or spherical reflective trackers are snapped onto metal posts projecting upwardly from the tracking array.

Often, there is a need to replace one or more defective trackers attached to the tracking array while a navigation assisted or robotically assisted surgery is being performed. Removing the trackers by hand needs to be done very carefully so as not to disturb the tracking array itself. If there is sufficient movement of the tracking array, it may cause the system to lose tracking registration and consequently, the entire time-consuming registration of the tracking array relative to the patient needs to be redone.

Accordingly, there is a need for an improved apparatus and method for removing the trackers without disturbing the tracking array which is already attached to the patient body.

### SUMMARY

A tool for removing circular trackers attached to tracking array posts. The tool includes a tubular body and a driver tube sized to be received in the tubular body. The distal portion of the tubular body and driver tube has respective first and second apertures that are circumferentially partially open in a lateral direction for receiving the circular tracker. The two apertures cooperate

with each other such that when the second aperture is rotated relative to the first aperture, the driver tube slides upwardly relative to the tubular body so as to lift the circular tracker from the post.

Since the pulling force is applied to the circular tracker relative to the tubular body, the tracker is removed without any force applied to the tracking array so as to minimize the chance of losing registration. Also, since the two apertures close to capture the tracker, the chance of losing the removed tracker in the patient body is substantially reduced.

### BRIEF DESCRIPTION OF THE DRAWINGS

FIG. 1 is a perspective view of both parts in same orientation.

FIG. 2A is a side view of the main tubular body showing the side aperture in full 180 degrees.

FIG. 2B is a side view of the main tubular body at 90 degree offset from that of FIG. 2A to show the side aperture pointing to the left.

FIG. 3 is a bottom view of the tubular body of FIG. 2A.

FIG. 4A is a side view of the driver tube showing the side aperture in full 180 degrees.

FIG. 4B is a side view of the driver tube at 90 degree offset from that of FIG. 4A to show the side aperture pointing to the left.

FIG. 5 is a bottom view of the driver tube inside the tubular body of FIG. 4A.

FIG. 6 is a bottom view of the driver tube inside the tubular body showing a closed aperture.

FIG. 7 is a side view of a tracking array with a spherical tracker.

### DETAILED DESCRIPTION OF THE ILLUSTRATED EMBODIMENTS

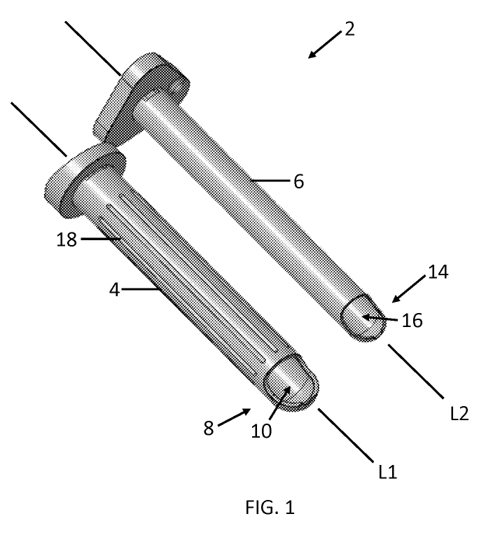

FIG. 1 illustrates a perspective view of a removal tool 2 for removing circular trackers 12 that are attached to a tracking array 38 according to one aspect of the present invention. The removal tool 2 includes a tubular body 4 and a driver tube 6. The distal portion 8 of the tubular body 4 has a first aperture 10 which is circumferentially partially open (e.g., 180 degrees as shown) in lateral and longitudinal (downward) directions relative to a longitudinal axis L1 defined by the tube. The first aperture 10 receives the circular tracker 12 to be removed as will be explained in more detail later herein. Longitudinal grooves 18 formed on the exterior surface of the tubular body 4 provides extra grip for holding the body by a user’s hand. The grooves 18 are circumferentially and uniformly spaced from each other.

The driver tube 6 is sized to be slidably received in the tubular body 4. Its distal portion 14 has a second aperture 16, which is also circumferentially partially open (e.g., 180 degrees as shown) in lateral and longitudinal (downward) directions relative to its longitudinal axis L2, the first and second apertures 8,14 cooperate with each other such that when the second aperture 16 is rotated relative to the first aperture 10, the driver tube 6 slides upwardly to pull the circular tracker off the post 36 of the tracking array 38 and simultaneously encloses the tracker within the driver and the tubular body 4 for removal.

The tubular body 4 at its proximal portion includes a flange 34 having a ramped surface 20 and the driver tube 6 at its proximal portion includes a triangle-shaped flange 22. The flange 22 of the driver tube 6 rides on the ramped surface 20 of the flange 34 to slide upwardly upon rotation of the driver tube 6 relative to the tubular body 4. The sliding movement causes the driver tube 6 to pull the circular tracker from the post 36. In the embodiment shown, a downwardly extending partially round projection 24 of the flange 22 is adapted to ride on the ramped surface 20.

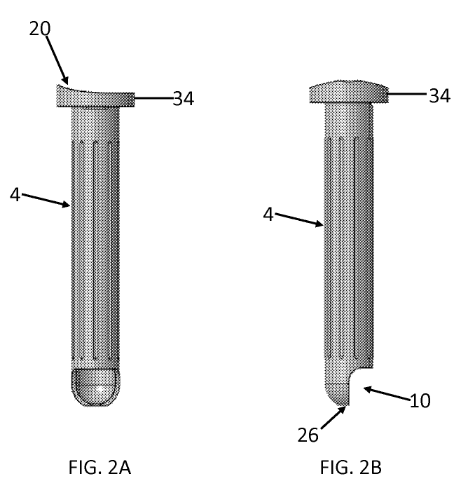

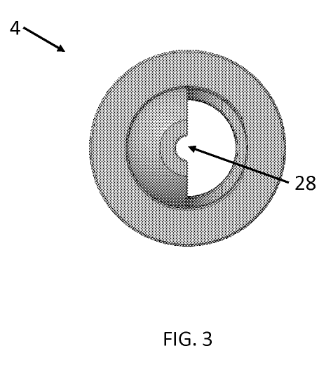

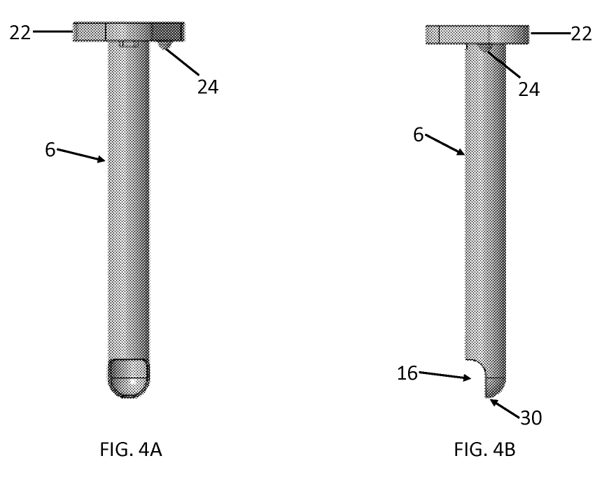

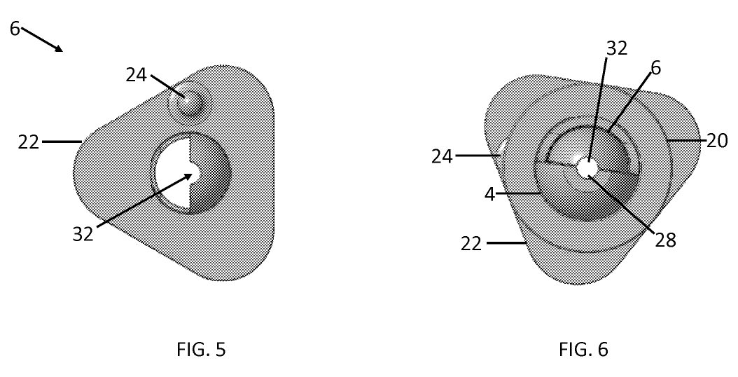

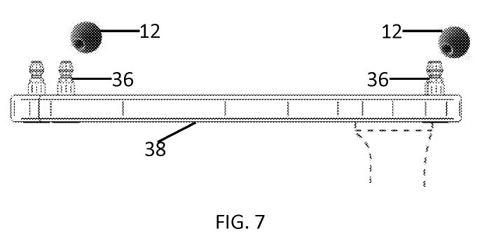

The distal portion 8 of the tubular body 4 as shown in FIGS. 2A-3 includes a partially open bottom surface 26 which has a central recess 28 adapted for partially surrounding the post 36. Similarly, the distal portion 14 of the driver tube 6 also has a partially open bottom surface 30 having a central recess 32 adapted for partially surrounding the post 36.

The ramped surface 20 is shaped such that rotation of the driver tube 6 in either direction causes the driver tube 6 to slide upwardly as the projection 24 rotates toward the highest point of the ramped surface 20. When the projection 24 is resting on the highest point of the ramped surface 20, the first and second apertures 10,14 face each other to enclose the circular tracker 12 as shown in FIG. 6.

The aperture of each of the tubular body 4 and the driver tube 6 is circumferentially open by at least 150 degrees. As shown, the apertures are open 180 degrees circumferentially.

In operation, the driver tube 6 is inserted into the tubular body 4 until the projection 24 of the flange 22 bottoms out and contacts the ramped surface 20. The projection 24 should be rotated away from the highest point in the ramped surface 20 until the first and second apertures 10,14 are nested and aligned so that they are pointed in the same direction.

The nested distal portions 8,14 of the tool 2 are then placed on the side of the circular tracker 12 to be removed. The tracker 12 is attached to the post 36 of the tracking array 38 which itself is attached to a patient bone. While the central recesses 28,32 contact the post 36, the driver tube 6 is rotated. As the driver tube 6 rotates, the tracker 12 is pulled off the post 36 and is captured by the closed apertures. Since the tracker 12 is pulled by the driver tube 6 relative to the tubular body 4, the tracker 12 can be removed without any force being applied to the tracking array 38. Also, closing of the apertures from rotation of the driver tube 6 avoids dropping the tracker 12 into the surgical site. The removed tracker 12 can then be discarded away from the surgical site.

While it is apparent that the invention disclosed herein is well calculated to fulfill the objects stated above, it will be appreciated that numerous modifications and embodiments may be devised by those skilled in the art. While devices and methods are described in terms of “comprising,” “containing,” “having,” or “including” various elements or steps, the devices and methods can also “consist essentially of” or “consist of” the various elements and steps.

What is claimed is:

1. A removal tool for a circular tracker attached to
a post of a tracking array comprising:
a tubular body including a distal portion having a first aperture, the first aperture being circumferentially partially open in a lateral direction and partially open in a downward direction for receiving the circular tracker; and
a driver sized to be slidably received in the tubular body and including a distal portion having a second aperture, the second aperture being circumferentially partially open in a lateral direction and partially open in a downward direction, one of the tubular body and the driver having a ramped surface adapted to engage with the other of the tubular body and the driver such that when the second aperture is rotated relative to the first aperture, the circular tracker is enclosed within the driver and the tubular body and is lifted from the post by the sliding driver for removal.

2. The removal tool of claim 1, wherein the tubular body includes the ramped surface and a portion of the driver rides on the ramped surface to slide upwardly upon rotation of the driver relative to the tubular body.

3. The removal tool of claim 1, wherein the tubular body includes the ramped surface and the driver includes a flange adapted to rotate against the ramped surface such that the driver slides upwardly to pull the attached circular tracker from the post.

4. The removal tool of claim 2, wherein the flange includes a downwardly extending projection adapted to ride on the ramped surface.

5. The removal tool of claim 1, wherein the distal portion has a bottom surface having a central recess adapted for partially surrounding the post.

6. The removal tool of claim 1, wherein each distal portion of the driver and the tubular body has a bottom surface having a central recess adapted for partially surrounding the post.

7. The removal tool of claim 1, wherein the tubular body has a flange having the ramped upper surface for engagement with the driver.

8. The removal tool of claim 1, wherein the tubular body has a flange having the ramped surface and the driver includes a flange whose bottom surface is adapted to rotate against the ramped surface such that the driver slides upwardly to pull the circular tracker from the post upon rotation of the driver relative to the tubular body in either circumferential direction.
